(19) United States
(12) Patent Application Publication
(10) Pub. No.: US 2017/0269061 A1
(43) Pub. Date: Sep. 21, 2017

(54) METHOD FOR ASSESSING SENSITIVITY OF A TARGET DRUG IN VITRO AND A BIOMARKER USED THEREOF

(71) Applicants: Tunghai University, Taichung City (TW); CHENG CHING HOSPITAL, Taichung City (TW)

(72) Inventors: Wei-Ting CHAO, Tiachung (TW); King-Jen CHANG, Taichung (TW); Yuan-Chiang CHUNG, Taichung (TW)

(21) Appl. No.: 15/076,456

(22) Filed: Mar. 21, 2016

(51) Int. Cl. G01N 33/50; G01N 33/574

(52) U.S. Cl. CPC G01N 33/5011; G01N 33/57415; G01N 2333/47

(57) ABSTRACT

This present invention is to provide a method for assessing sensitivity of target drug in vitro and a biomarker used thereof. It is to identify sensitivity of a target drug by analyzing expression levels of CAV-1 and Her-2 in a tissues sample provided by a subject to achieve effect of personal and precise medical treatment.

### BACKGROUND OF THE INVENTION

1. Field of the Invention

This invention relates to a biomarker and application thereof, specially relates to a method for assessing sensitivity of a target drug in vitro and a biomarker used thereof.

2. Description of the Related Art

Human epidermal growth factor receptor 2 (HER-2) has been identified as oncoprotein in breast cancer. The overexpression of HER-2 mRNA and protein occurs in 20-30% of invasive breast cancers and is a predictor of poor clinical outcome. The humanized monoclonal antibody, trastuzumab (Herceptin), binds to the extramembrane domain of HER-2 to inhibit the proliferation and survival of HER-2 dependent tumors. After several effective trials, in 2001, trastuzumab was approved by the Food and Drug Administration (FDA) in the USA for patients with advanced breast cancers that express HER-2. Despite the success of this therapeutic treatment, naked trastuzumab targeting of HER-2 expression in breast cancer is rarely curative by itself, and most of the effects of this drug are achieved in combination with chemotherapy. However, there are adverse effects of combination therapy: 27% of patients treated concurrently with trastuzumab and anthracyclines, and 13% with trastuzumab and paclitaxed, had cardiotoxic side effects

Recent advances in antibody drug conjugate (ADC) techniques allow for the linkage of specific monoclonal antibodies with potent cytotoxic drugs to reduce systemic toxicity and increase therapeutic benefits in cancer patients. HER-2-based ADC targeting have been investigated for clinical application in breast cancer treatment using trastuzumab emtasime (trastuzumab-DM1; T-DM1), in which trastuzumab is conjugated through a stable thioether bond to the maytansanoid derivative emtasine. The latter has potent anti-mitotic effects by preventing microtubule assembly. The antibody portion of the conjugate binds to the HER-2 receptor on the surface of cancer cells allowing for the internalization of T-DM1 and its subsequent degradation to release the lethal drug. Under this mechanism, T-DM1 treatment allows patients a better quality of life by preventing the toxic effects of microtubule targeting chemotherapy. The National Comprehensive Cancer Network Guidelines (NCCN) approved T-DM1 application in metastatic HER-2 positive breast cancer in 2013. Despite the efficacy of T-DM1, most patients treated with this ADC eventually progress to more serious stages of disease and some HER-2 positive breast cancers are primarily non-responsive or only have a minimal response to T-DM1.

The target drug is the current trend for treating cancer, but the cost of the target drug is too expensive. Most of the patients can't afford it for the long-term treatment. Even though the patient can afford the cost of the target drug, not all of the patient response well to the target drug. Therefore, deciphering the mechanism of T-DM1 internalization is critical for its clinical application in advanced precision therapy.

### SUMMARY OF THE INVENTION

The major propose of this present invention is to provide a biomarker for assessing drug sensitivity in vitro, caveolin-1 (CAV-1). It is for predict drug sensitivity to achieve the effect to predict prognosis of patient or development of cancer target drug.

Another purpose of this present invention is to provide a method for assessing sensitivity of target drug to a breast cancer patient in vitro. It is to identify sensitivity of a target drug by analyzing expression levels of CAV-1 and Her-2 in breast cancer tissues provided by a patient to achieve effect of personal and precise medical treatment.

In order to achieve the foresaid purposes, this present invention provides a biomarker for assessing drug sensitivity in vitro which is caveolin-1. By identifying expression level of caveolin-1, the sensitivity of cancer target drug can be further determined.

Preferably, the cancer is breast cancer.

Preferably, the target drug is trastuzumab emtansine (T-DM1).

In a preferable embodiment, this present invention is to provide a method for assessing sensitivity of target drug in vitro by obtaining a tissue sample from a subject, and determining expression of caveolin-1 in the tissue sample, wherein when the expression of caveolin-1 in the tissue sample is higher than expression of caveolin-1 in a control sample, the subject is having sensitivity to the cancer target drug.

Preferably, the breast cancer tissue sample expresses the Her2.

Preferably, the target drug is an antibody-drug conjugate (ADC), wherein, T-DM1 is preferable.

More specifically, this present invention of the method for assessing sensitivity of target drug to a breast cancer patient in vitro includes following steps:

step A: obtaining a normal tissue sample and a Her2-positive breast cancer tissue sample from the subject;

step B: determining expressions of caveolin-1 of the normal tissue sample and the Her2-positive breast cancer tissue sample from the subject;

step C: comparing the expressions of caveolin-1 in the Her2-positive breast cancer tissue with the expressions of caveolin-1 in the normal tissue sample, wherein greater levels of caveolin-1 in the Her-2 positive breast cancer tissue as compared to the normal tissue sample is indicative of sensitivity to the target drug in the subject.

Preferably, step B is using immunohistochemistry method to determine the expression caveolin-1 in each of the samples.

Preferably, step B is using protein quantification method to determine the expression of caveolin-1 in each of the samples.

### BRIEF DESCRIPTION OF THE DRAWINGS

FIG. 1A shows negative staining of caveolin-1 in breast cancer tissues by immunohistochemical staining.

FIG. 1B shows weak staining of caveolin-1 in breast cancer tissues by immunohistochemical staining.

FIG. 1C shows moderate staining of caveolin-1 in breast cancer tissues by immunohistochemical staining.

FIG. 1D shows heavy staining of caveolin-1 in breast cancer tissues by immunohistochemical staining, wherein the scale bar is 100 μm.

FIG. 2 shows the results of cell lysates of breast cancer cell lines, ZR-75-1, MDA-MB-231, MCF-7, SKBR-3, and BT-474 with antibodies against caveolin-1 and clathrin by western blot analysis. GAPDH was used as an internal control.

FIG. 3 shows the expression of caveolin-1 in SKBR-3, and BT-474 cells.

FIG. 4 shows the quantification result l of caveolin-1 expression in SKBR-3, and BT-474 cells.

FIG. 5 shows the expression of clathrin in SKBR-3, and BT-474 cells.

FIG. 6 shows the quantification result of clathrin expression in SKBR-3, and BT-474 cells.

FIG. 7 shows the quantification result of caveolin-1 expression in different breast cancer cells.

FIG. 8 shows the quantification result of clathrin expression in different breast cancer cells.

FIG. 9 shows the results of detecting caveolin-1 in MDA-MB-231, SKBR-3, and BT-474 cells which incubated with trastuzumab, respectively, by immuno-confocal microscopy. Green color indicates trastuzumab, red color indicates caveolin-1.

FIG. 10 shows the results of detecting caveolin-1 in MDA-MB-231, SKBR-3, and BT-474 cells which incubated with T-DM1, respectively, by immuno-confocal microscopy. Green color indicates T-DM1, red color indicates caveolin-1.

FIG. 11 shows the phase contrast images of MDA-MB-231, SKBR-3, and BT-474 cells were treated with trastuzumab, T-DM1 or paclitaxel for 72 hours.

FIG. 12 shows the cell survival graph.

FIG. 13 shows the expression of the pro-survival marker Bcl-2 in MDA-MB-231, SKBR-3, and BT-474 cells by Western blot analysis.

FIG. 14 shows the quantification result of the pro-survival marker Bcl-2 in MDA-MB-231, SKBR-3, and BT-474 cells.

FIG. 15 shows the staining results of MDA-MB-231, SKBR-3, and BT-474 cells were treated with T-DM1 by inverted fluorescence microscope. Green color indicates annexin V positive apoptotic cell, red color indicates PI positive necrotic cell (arrows).

FIG. 16 shows the quantification result of cell apoptosis was determined by annexin V staining.

FIG. 17 shows the quantification result of annexin V positive/PI negative apoptosis from FIG. 15.

FIG. 18 shows the cell viability of MDA-MB-231, SKBR-3 and BT-474 cells.

FIG. 19 shows the overexpression and knockdown of caveolin-1 in BT-474 and SKBR-3 cells by Western blot analysis. BT indicated BT-474 cell; SK indicated SKBR-3 cell; si-CAV-1 indicated caveolin-1 siRNA. Arrow indicated overexpression of GFP tagged caveolin-1.

FIG. 20 shows the quantification result of Caveolin-1 expression in SKBR-3 cells transfected with caveolin-1 siRNA by Western blot analysis.

FIG. 21 shows the expression of the pro-survival marker Bcl-2 by Western blot analysis. BT indicated BT-474 cell; SK indicated SKBR-3 cell; si-CAV-1 indicated caveolin-1 siRNA. Arrow indicated overexpression of GFP tagged caveolin-1.

FIG. 22 shows the cell viability after the cells treating with treated with T-DM1 for 72 hours.

FIG. 23 shows the quantification result of the pro-survival marker Bcl-2 from FIG. 21.

FIG. 24 shows the quantification result of cell apoptosis.

### DETAILED DESCRIPTION OF THE INVENTION

All data from the following examples are expressed as mean±standard deviation. A Student t-test was used to compare continuous variables. Differences at the P<0.05, P<0.01 and P<0.001 were considered statistically significant. Tukey Kramer test was used for multiple comparisons, different letters represent significant difference at the P<0.01 level.

### Example 1: Collecting Breast Cancer Tissue Samples

Breast epithelium tissues were collected for 32 female breast cancer patients, age range 35-85 years old and median age 48.5 years old, who had undergone resection for localized breast cancer between April 2011 and December 2013 at Cheng-Ching General Hospital, Taiwan. Wherein, there were three patients in stage I, 13 patients in stage II, and 16 patients in stage III of breast cancer. The collection and use of these samples were reviewed and approved by the Cheng-Ching General Hospital Institutional Review Board (HT110018) and the written informed consent was obtained from all patients.

### Example 2: Immunohistochemistry Analysis

The breast cancer tissue from each of the patient was sectioned, and then incubated with anti-caveolin-1 (1:100) or anti-HER-2 (1:1000) antibodies (Dako, Carpinteria, Calif., USA) at room temperature for 1 hr. A histostain-SP diaminobenzidine tetrahydrochloride (DAB) kit (Invitrogen, ThermoFisher, Waltham, Mass., USA) was then used to visualize the primary antibody.

For HER-2, the strength of the membranous staining was graded from −, +, ++, +++ and only +++ was considered positive according to HercepTest guideline from Dako.

The staining strength of the scoring used for caveolin-1 (hereinafter “CAV-1”) by immunohistochemistry was the “I” index; the equation for which is:

I=0×f0+1×f1+2×f2+3×f3,

wherein f0-f3 are the fractions of the cells showing a defined level of staining intensity (from 0-3).

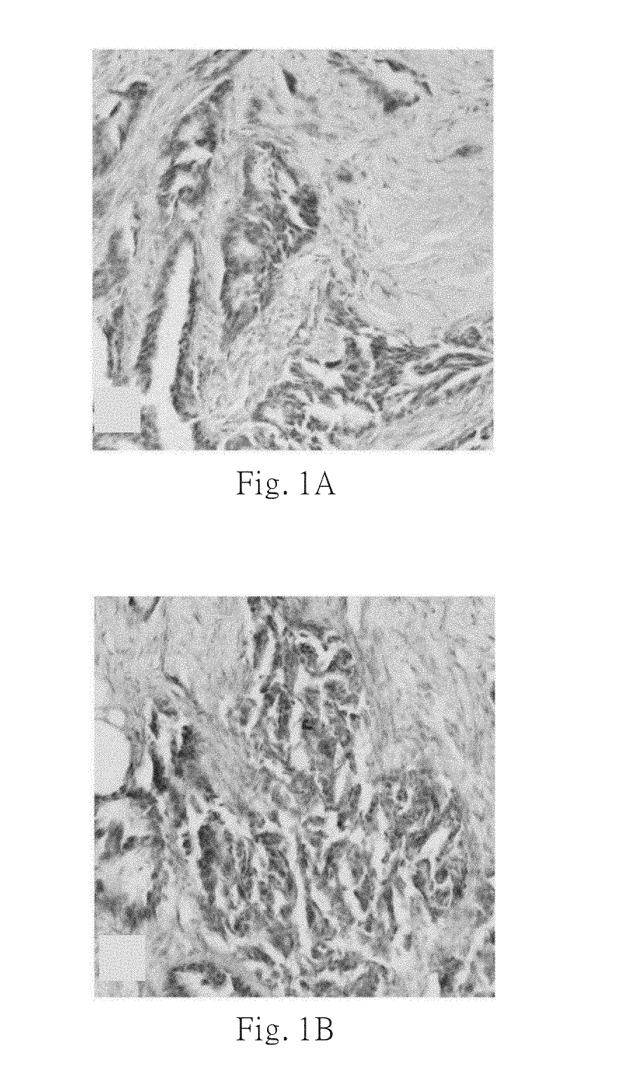

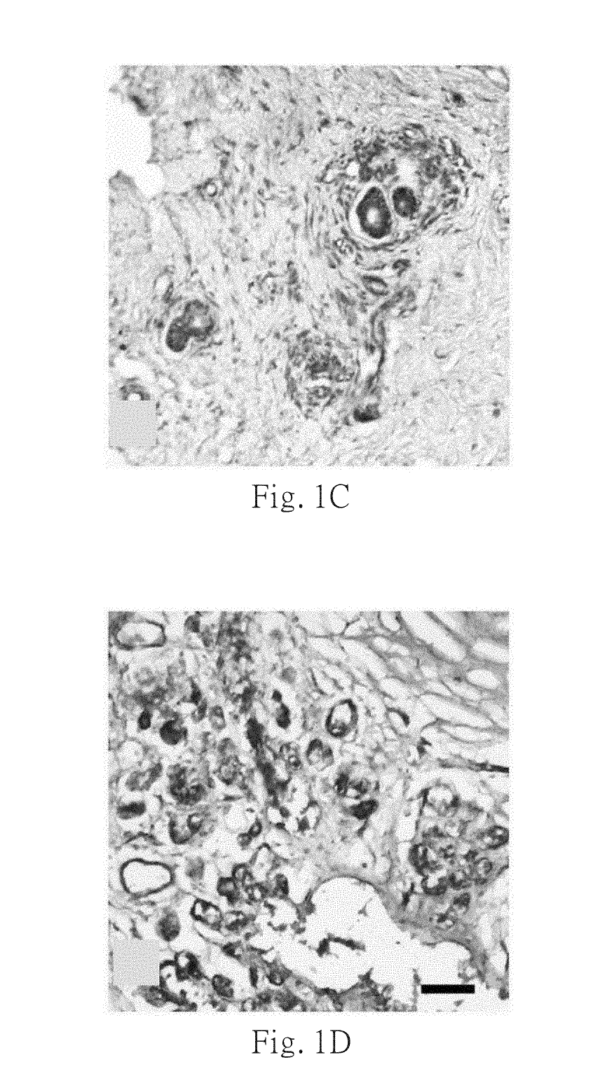

Please see the FIG. 1, the numbers 0-3 represent the following:

“0” negative, no detectable staining, as showed in FIG. 1A;

“1” weak, but still detectable staining, as showed in FIG. 1B;

“2” moderate, clearly positive but still weak, as showed in FIG. 1C; and

“3” heavy and intense staining, as showed in FIG. 1D.

Therefore, scores range from 0-3. When a score greater than the mean value of 1.5 was considered as high expression, and a score less than the mean value of 1.5 was considered as low expression of CAV-1.

According to the above-mentioned method, the immunohistochemistry analysis was performed on each of the breast epithelium tissues gathered from example 1, including tumor site and non-tumor site (stroma). The result is shown as Table 1. And the result of Table 1 was statistically analyzed to generate the result shown as Table 2. Furthermore, Table 3 shows the analysis result of the association of CAV-1 expression with HER-2, estrogen receptor (ER) or progesterone receptor (PR) in patients by immunohistochemistry. Wherein, the expression results of CAV-1 are expressed as mean±standard deviation. In each table, the symbol (+) indicate the positive expression according to the clinical reports from pathology department in Cheng-Ching hospital and the “I index” of CAV-1 greater than median value 1.5 was considered as positive.

TABLE 1
Expression of HER-2, ER (estrogen receptor), PR (progesterone
receptor) and Cav-1 (caveolin-1) in 32 breast cancer patients

| | | | | | | | CAV-1 | CAV-1 |
|---|---|---|---|---|---|---|---|---|
| Patient | Age | Stage | ER | PR | HER-2 | Grade | Tumor | Stroma |
| 1 | 55 | 2 | − | − | − | 3 | 0.5 | 1.0 |
| 2 | 44 | 2 | − | − | − | 3 | 1.4 | 1.4 |
| 3 | 42 | 3 | + | − | − | 3 | 1.8 | 1.7 |
| 4 | 49 | 2 | + | + | + | 2 | 2.7 | 2.3 |
| 5 | 70 | 3 | + | + | − | 2 | 0.3 | 0.8 |
| 6 | 42 | 3 | − | − | + | 3 | 1.6 | 1.8 |
| 7 | 35 | 2 | + | + | + | 2 | 0.3 | 1.1 |
| 8 | 47 | 3 | + | + | + | 2 | 1.6 | 1.4 |
| 9 | 44 | 2 | + | + | − | 2 | 1.9 | 1.5 |
| 10 | 47 | 3 | + | + | − | 2 | 1.6 | 1.4 |
| 11 | 41 | 2 | + | + | − | 2 | 2.6 | 1.4 |
| 12 | 55 | 1 | + | + | − | 1 | 1.4 | 1.4 |
| 13 | 85 | 3 | − | − | + | 3 | 1.8 | 1.6 |
| 14 | 68 | 2 | + | − | − | 2 | 2.7 | 1.8 |
| 15 | 50 | 3 | + | + | − | 2 | 2.4 | 2.0 |
| 16 | 39 | 3 | + | + | − | 2 | 1.8 | 1.6 |
| 17 | 48 | 2 | + | + | + | 2 | 2.8 | 1.6 |
| 18 | 55 | 2 | − | − | + | 2 | 2.4 | 2.7 |
| 19 | 61 | 2 | + | + | − | 2 | 1.4 | 1.4 |
| 20 | 40 | 3 | + | + | − | 3 | 1.9 | 1.4 |
| 21 | 55 | 3 | − | + | − | 3 | 2.9 | 2.1 |
| 22 | 47 | 1 | + | + | − | 1 | 2.4 | 2.2 |
| 23 | 48 | 2 | + | + | − | 2 | 0.7 | 1.9 |
| 24 | 44 | 3 | + | + | − | 1 | 1.4 | 1.8 |
| 25 | 72 | 3 | + | − | − | 3 | 2.9 | 1.7 |
| 26 | 70 | 3 | − | − | + | 3 | 2 | 1.8 |
| 27 | 33 | 2 | − | − | + | 3 | 2.4 | 1.7 |
| 28 | 59 | 2 | − | − | + | 3 | 1.7 | 1.0 |
| 29 | 45 | 3 | + | − | − | 3 | 1.1 | 1.5 |
| 30 | 63 | 1 | + | − | − | 3 | 1.4 | 1.2 |
| 31 | 63 | 3 | − | − | − | 3 | 2.6 | 1.2 |
| 32 | 56 | 3 | + | − | + | 3 | 2.4 | 1.7 |

TABLE 2
Statistically analysis result of table 1

| | | | | Tumor | Stroma |
|---|---|---|---|---|---|
| | HER (+) | ER (+) | PR (+) | CAV-1 (+) | CAV-1 (+) |
| Stage 1 | 0/3 | 3/3 | 2/3 | 1/3 | 1/3 |
| | (0%) | (100%) | (66.67%) | (33.33%) | (33.33%) |
| Stage 2 | 6/13 | 8/13 | 7/13 | 8/13 | 7/13 |
| | (46.15%) | (61.54%) | (53.85%) | (61.54%) | (53.85%) |
| Stage 3 | 5/16 | 11/16 | 8/16 | 13/16 | 11/16 |
| | (31.25%) | (68.75%) | (50%) | (81.25%) | (68.75%) |
| All | 11/32 | 22/32 | 17/32 | 22/32 | 19/32 |
| patient | (34.38%) | (68.75%) | (53.13%) | (68.75%) | (59.38%) |

TABLE 3
The association of CAV-1 expression with HER-2, ER or PR

| | CAV-1 expression (I index) | |
|---|---|---|
| HER-2 (+) | 1.97 ± 0.7 | p = 0.45 |
| HER-2 (−) | 1.77 ± 0.77 | |
| ER (+) | 1.80 ± 0.77 | p = 0.63 |
| ER (−) | 1.93 ± 0.69 | |
| PR (+) | 1.77 ± 0.82 | p = 0.59 |
| PR (−) | 1.91 ± 0.66 | |

After analysis, twenty-two cases (68.75%) of invasive ductal carcinoma showed positive expression (score 1.5) of CAV-1 staining, and negative expression of CAV-1 (score <1.5) was noted in 10 cases (31.25%).

Furthermore, the expression of CAV-1 in HER-2 (+) tissue was 1.97±0.70, the expression of CAV-1 in HER-2 (−) tissue was 1.77±0.77. There were not significantly different between HER-2 (+) and HER-2 (−) tissues (p=0.45). The expression of CAV-1 in the ER (+) tissue was 1.80±0.77, expression of CAV-1 in the ER (−) tissue was 1.93±0.69. There were not significantly different between ER (+) and ER (−) tissues (p=0.63). The expression of CAV-1 in the PR (+) tissue was 1.77±0.82, expression of CAV-1 in the PR (−) tissue was 1.91±0.66. There were not significantly different between PR (+) and PR (−) tissues (p=0.59).

### Example 3: Western Blot

In addition, according to the Western blot method to study the protein expression of each breast epithelium tissue, the result is shown as Table 4. 8 (72.72%) of the 11 HER-2 positive breast cancer patients were found to have a higher expression of CAV-1 in tumor tissue than in non-tumor tissue. Sample of non-tumor site specimens all showed high expression of CAV-1 with heterogeneous distribution in ductal epithelial.

TABLE 4
The result of CAV-1 expression by Western blot and the association
with HER-2, ER or PR

| | Cav-1 expression, tumor>normal |
|---|---|
| HER-2 (+) | 8/11 (72.7%) |
| HER-2(−) | 17/21 (81%) |
| ER (+) | 18/22 (81.9%) |
| ER (−) | 7/10 (70%) |
| PR (+) | 14/17 (82.4%) |
| PR (−) | 11/15 (73.3%) |

### Example 4: Culturing Breast Cancer Cell Lines

The triple negative breast cancer MDA-MB-231 cell line, and MCF-7 cell line were used as HER-2 negative cells, and SKBR-3, BT-474, and ZR-75-1 were used as HER2 positive cells.

Expression of ER and PR in each breast cancer cell line was estimated by Allred scores, which were (0, 0) in MDA-MB-231, (6, 6) in MCF-7, (0, 0) in SKBR-3, (0, 8) in BT-474 and (3, 4) in ZR-75-1 cells. Each of the cancer cell line grown in Dulbecco's modified Eagle's Medium (DMEM) supplemented with 10% calf serum, penicillin, and streptomycin (GIBCO-BRL, Gaithersburg, Md., USA) and was kept in an incubator with 5% CO₂ at 37° C.

### Example 5: Endocytic Protein Expression in Breast Cancer Cell Lines

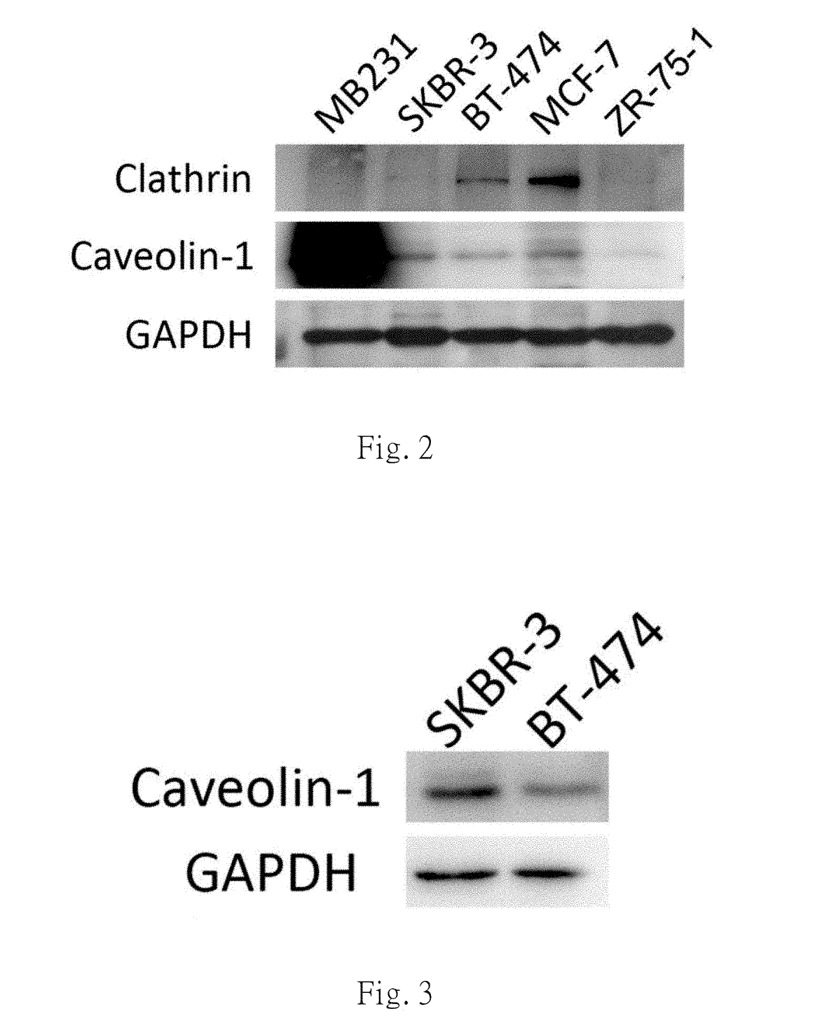

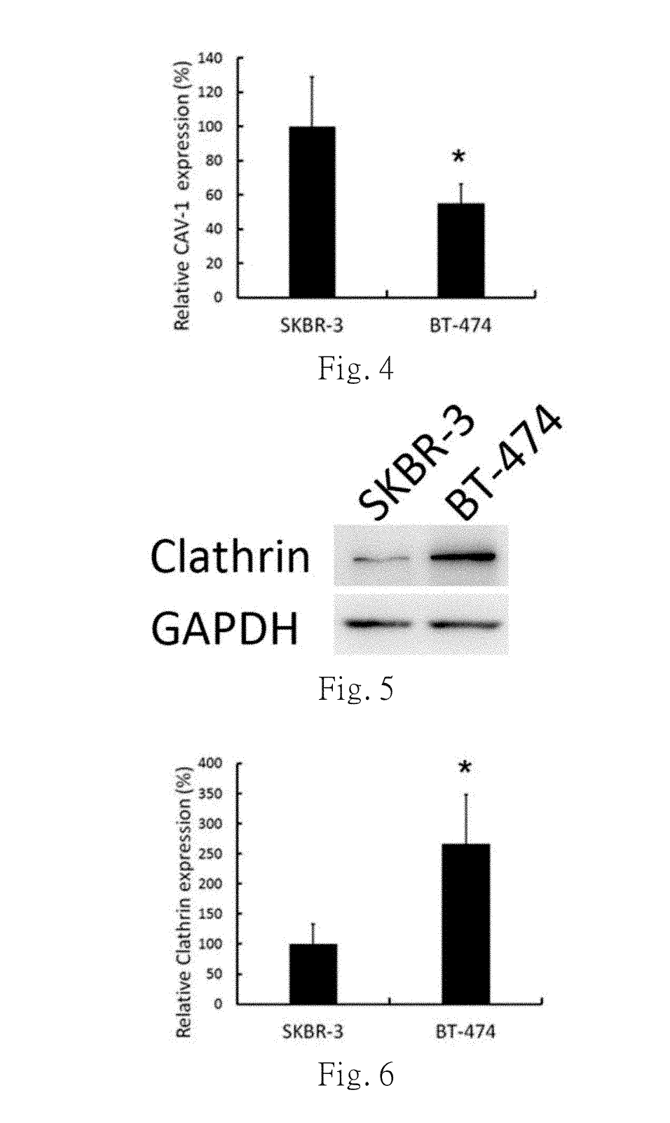

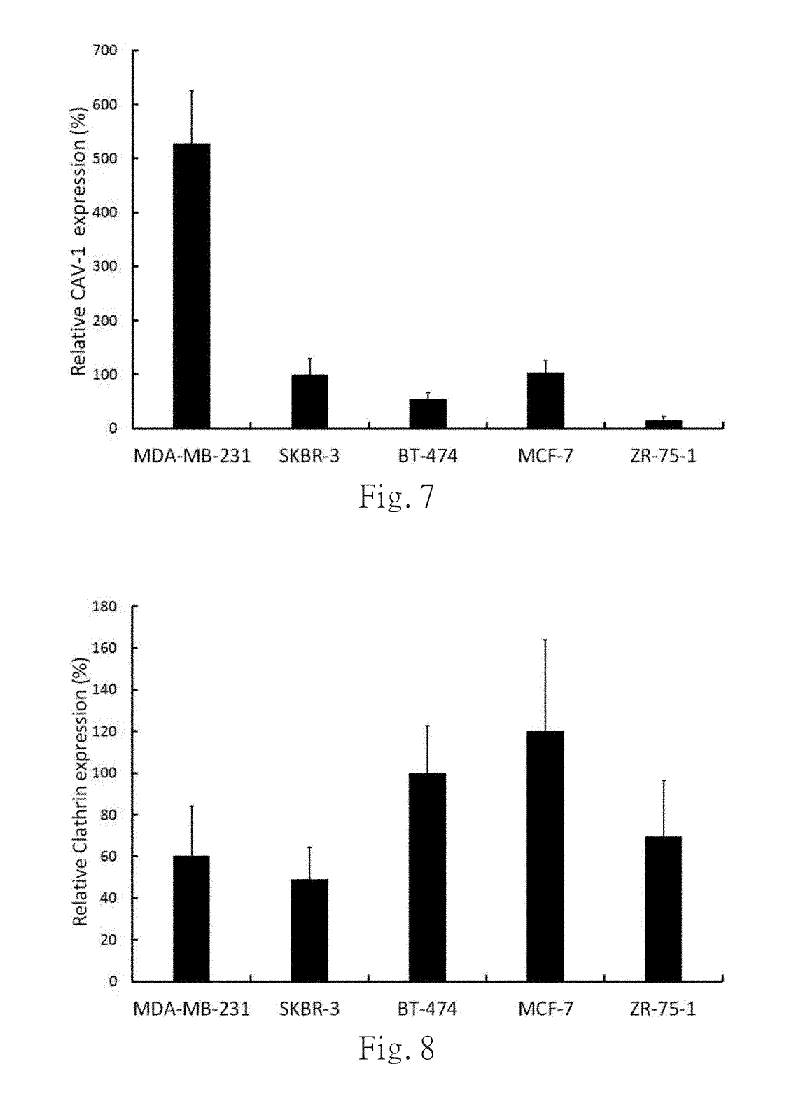

The expression of the two major endocytic proteins clathrin and caveolin-1 was examined by Western blotting in the breast cancer cell lines MDA-MB-231, MCF-7, SKBR-3, BT-474, and ZR-75-1, and the results were shown as FIGS. 2-8.

FIGS. 2-8 illustrate that caveolin-1 highly expressed in MDA-MB-231. Furthermore, for HER-2 positive cells, caveolin-1 expression was higher in SKBR-3 than in BT-474 cells. Clathrin was more abundant in BT-474 cells than in SKBR-3 cells. Triple negative MDA-MB-231, HER-2 positive (+++) SKBR-3, and BT-474 cell lines were therefore chosen for the following experiments.

### Example 6: Cell Experiment (I)

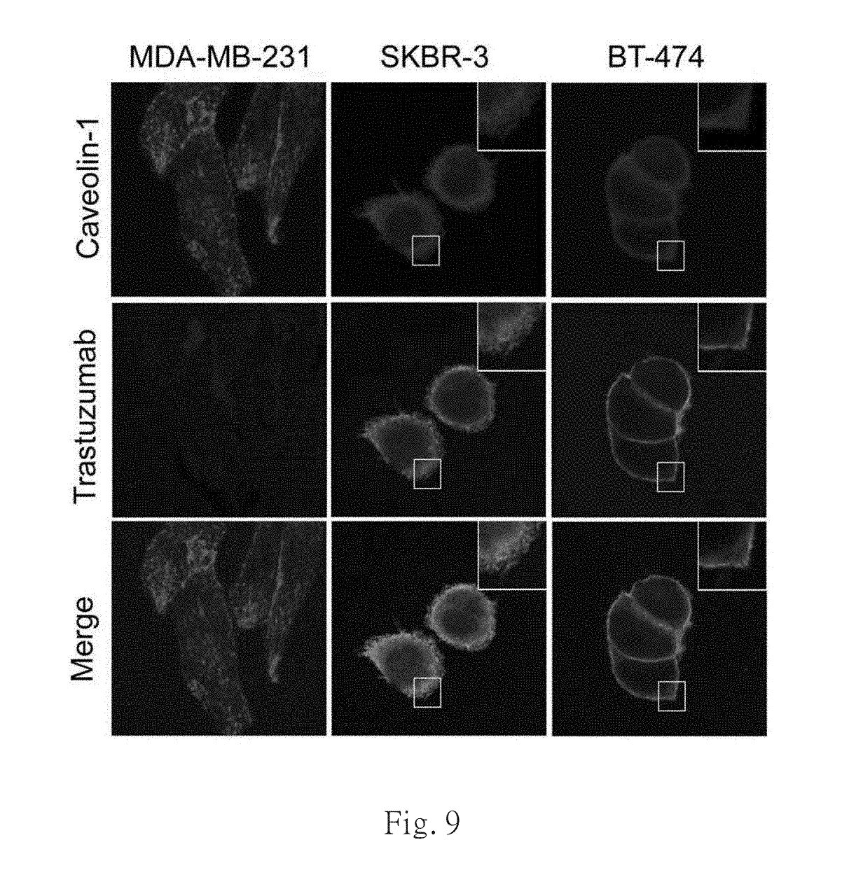

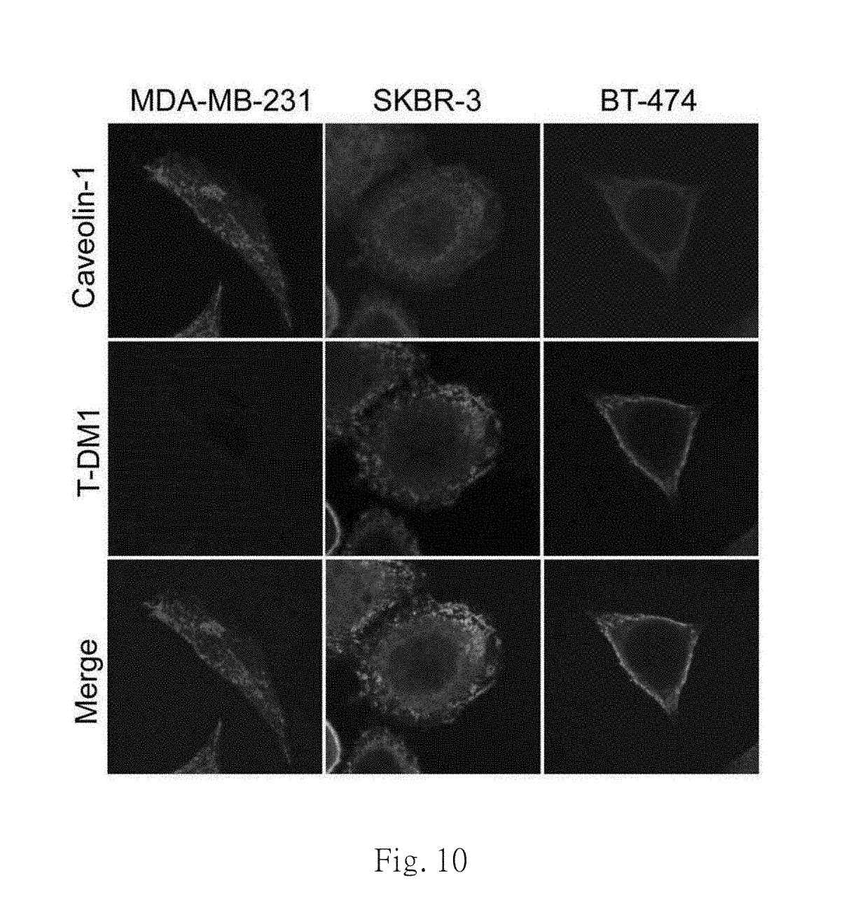

MDA-MB-231, SKBR-3 and BT-474 cells were respectively treated with T-DM1 (1 μg/ml) or trastuzumab (10 μg/ml) for 30 min. After washing, each of the cells was fixed with 3.7% formaldehyde and permeabilized with 0.1% Triton-X 100. The fixed cells were incubated with rabbit anti-caveolin-1 antibody (1:100 dilution in PBS/0.1% Triton-100/3% BSA) at room temperature for 1 hour and then incubated with Cy3-conjugated anti-rabbit secondary antibodies (1:200 dilution in PBS/0.1% Triton-100/3% BSA) at room temperature for 1 hour. The presence of trastuzumab and T-DM1 was revealed by a Cy2-conjugated anti-human secondary antibody. Expression of endocytic protein in each of the cells was observed by immunofluorescence microscopy, the results were shown as FIGS. 9 and 10.

Results from FIGS. 9 and 10 demonstrate that neither trastuzumab nor T-DM1 treated MDA-MB-231 cells clearly expressed caveolin-1. The intensity of CAV-1 staining was higher in SKBR-3 than BT-474 cells, and showed a clear cytosolic distribution. And, T-DM1 or trastuzumab were colocalized in the SKBR-3 cell membrane and cytosol. In BT-474 cells, most of the T-DM1 and trastuzumab was localized only in the cell membrane.

These results suggest that T-DM1 or trastuzumab were bound to HER-2 positive cells, and the internalization of trastuzumab and T-DM1 was associated with caveolin-1 expression.

### Example 7: Cell Experiment (II)

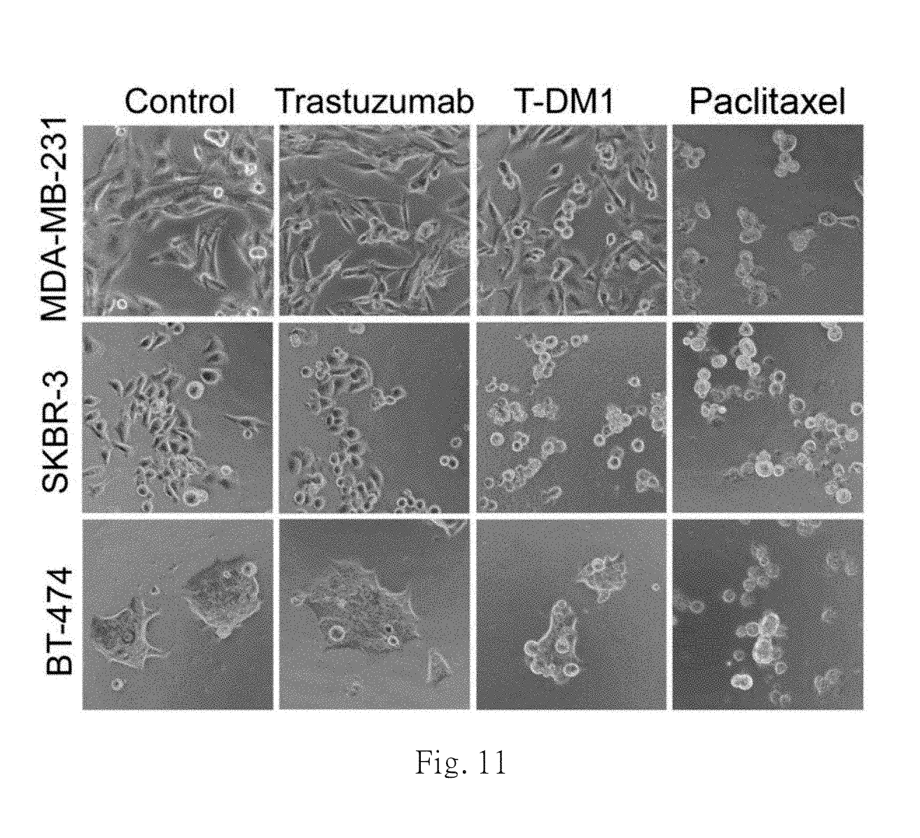

MDA-MB-231, SKBR-3 and BT-474 cells were respectively were seeded into plates at a density of 0.1×106 cells/ml and incubated alone or in 10 μg/ml trastuzumab (Herceptin, Roche Ltd., Basel, Swiss), 1 μg/ml T-DM1 (Kadcyla, Roche Ltd., Basel, Swiss), or 1 μg/ml paclitaxel (Phyxol, Sinphar Ltd., I-Lan, Taiwan) for 72 hour. Each of the cells under different conditions were observed by phase contrast microscope, the results were shown as FIG. 11.

Results from FIG. 11 demonstrate that paclitaxel would cause the most cytotoxicity to each of the cells.

### Example 8: Cell Viability Assay

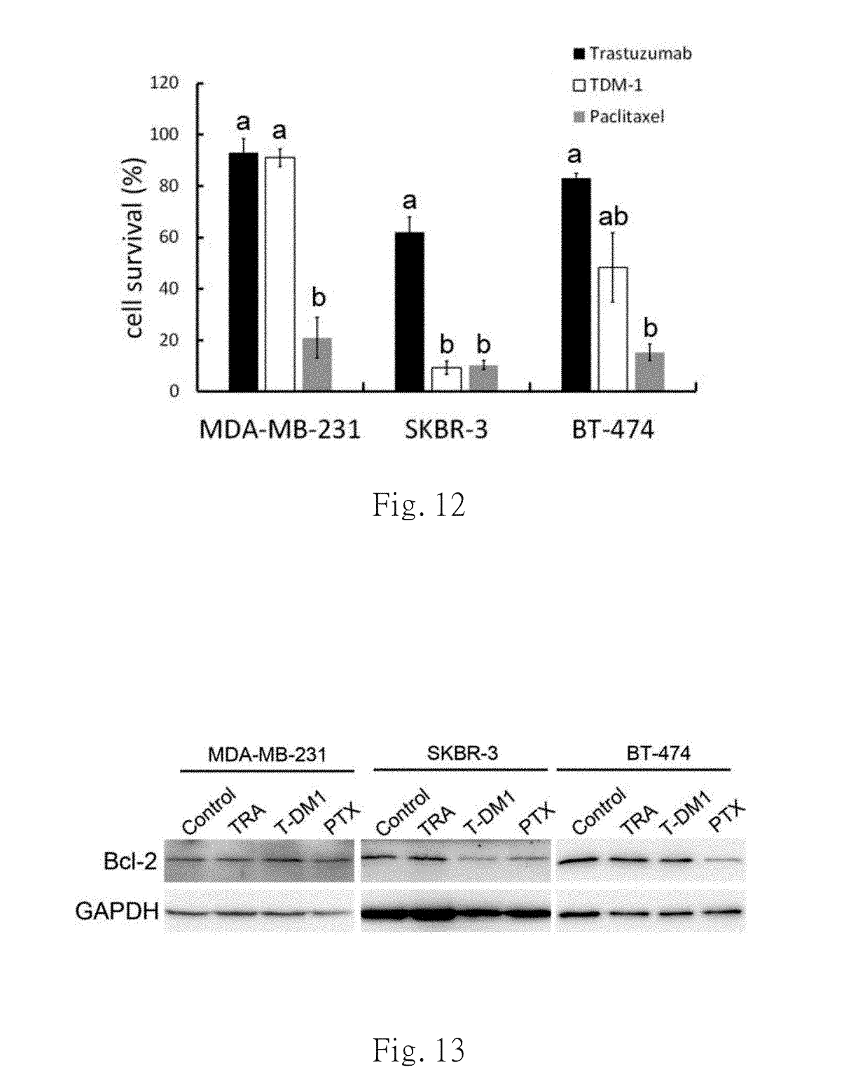

After culturing each of the cells under different condition in example 7, 0.1 ml of 0.4% trypan blue and deionized water (1:1) was added. Dead cells were stained blue, while live cells remained unstained. Therefore, the number of dead cells could be estimated and cell viability estimated with a hemocytometer. Wherein, the cell mortality was expressed as the percentage of trypan blue-positive cells compared to the total number of cells and the viability percentage was presented by the number of live cells divided by the number of untreated control cells×100. Results of cell viability were shown as FIG. 12.

The results of FIG. 12 show that SKBR-3 was significantly more sensitive to T-DM1 when compared to trastuzumab treatment (p<0.01) and the cytotoxic efficacy of T-DM1 was similar to paclitaxel treatment.

### Example 9: Cell Apoptosis Analysis

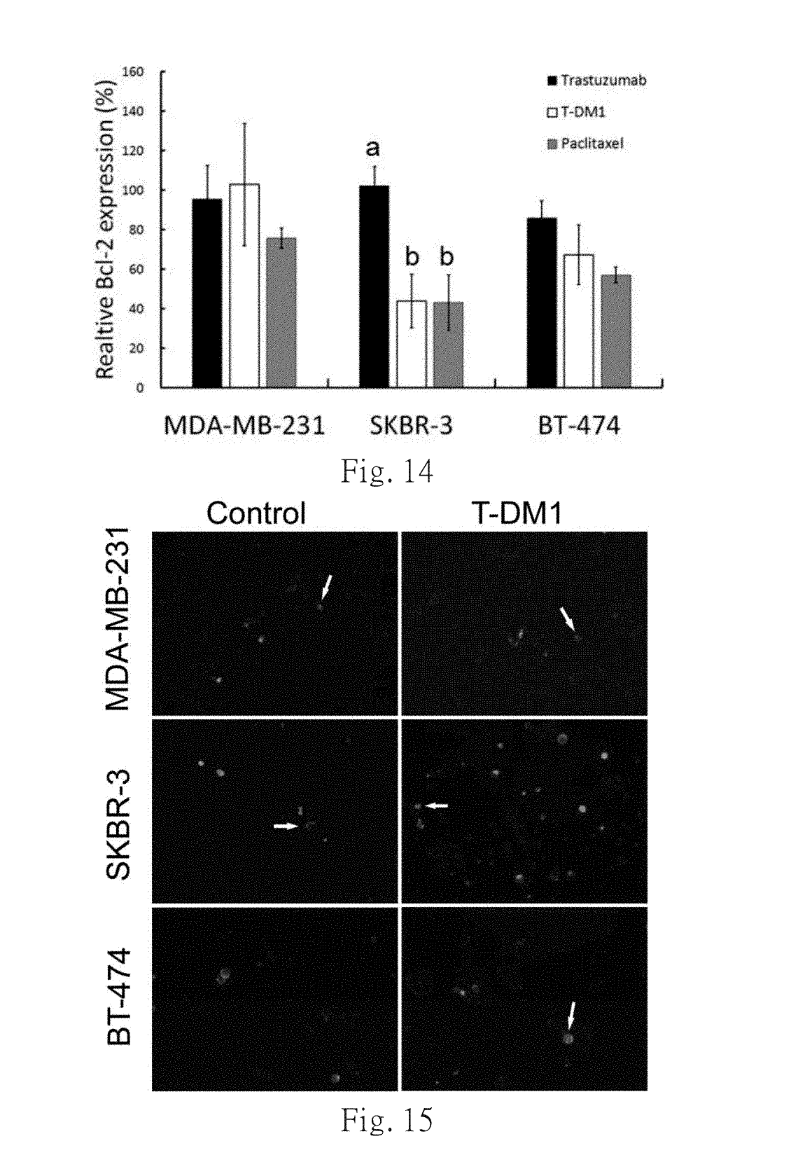

Pro-survival protein Bcl-2 expression in each of the cells, culturing under different condition in example 7 at 48 hours, were determined by Western blot. Results were shown as FIGS. 13 and 14.

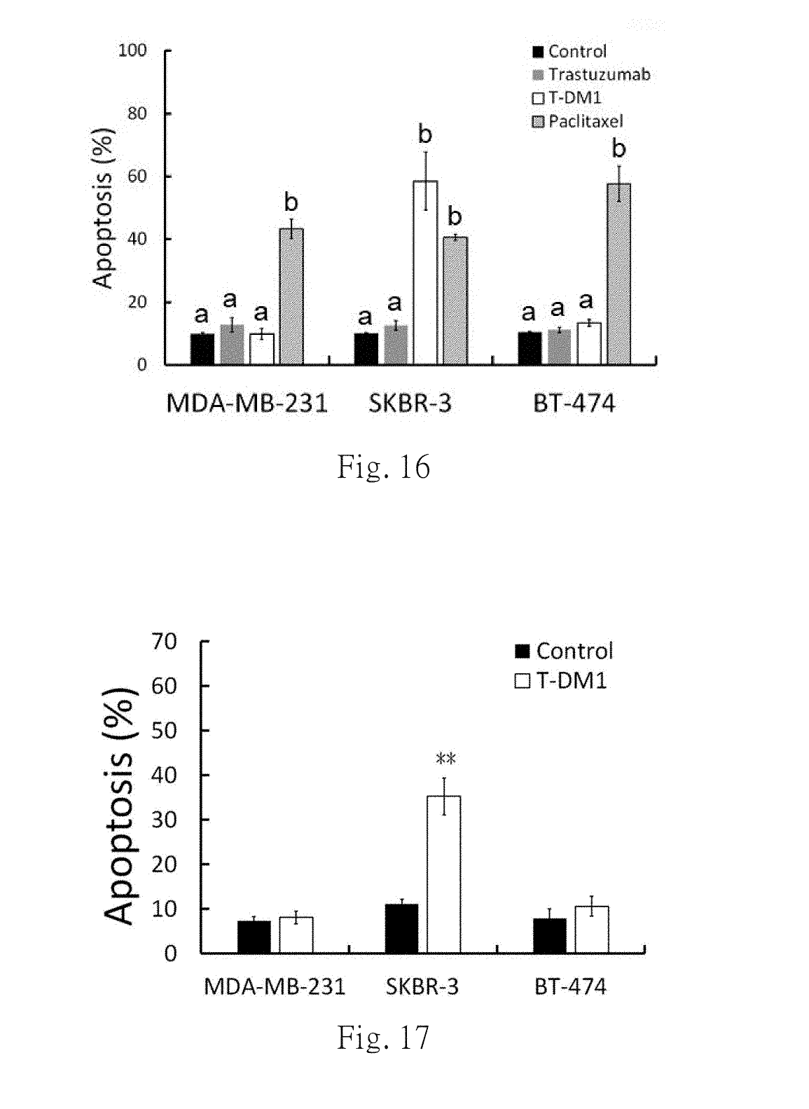

Using an annexin V detection kit (Invitrogen, ThermoFisher, Waltham, Mass., USA) to label cell surface phosphatidylserine of apoptotic cells and analysis these stained cells by flow cytometry. To further monitor the status of cell apoptosis, T-DM1 treated cells were live stained with annexin V and propodium iodide (PI) together. Co-staining images were taken by inverted fluorescence microscope shown in FIG. 15, wherein green color indicates annexin V positive apoptotic cell, red color indicates PI positive necrotic cell. Annexin V stained cells with PI positive staining were excluded as necrotic cells, apoptotic cells with only annexin V staining were counted and divided by total cells as the percentage of apoptosis. The percentage of apoptosis of each of the cells was shown as FIG. 16. And, annexin V positive/PI negative apoptosis in FIG. 15 was quantified. Results were shown at FIG. 17.

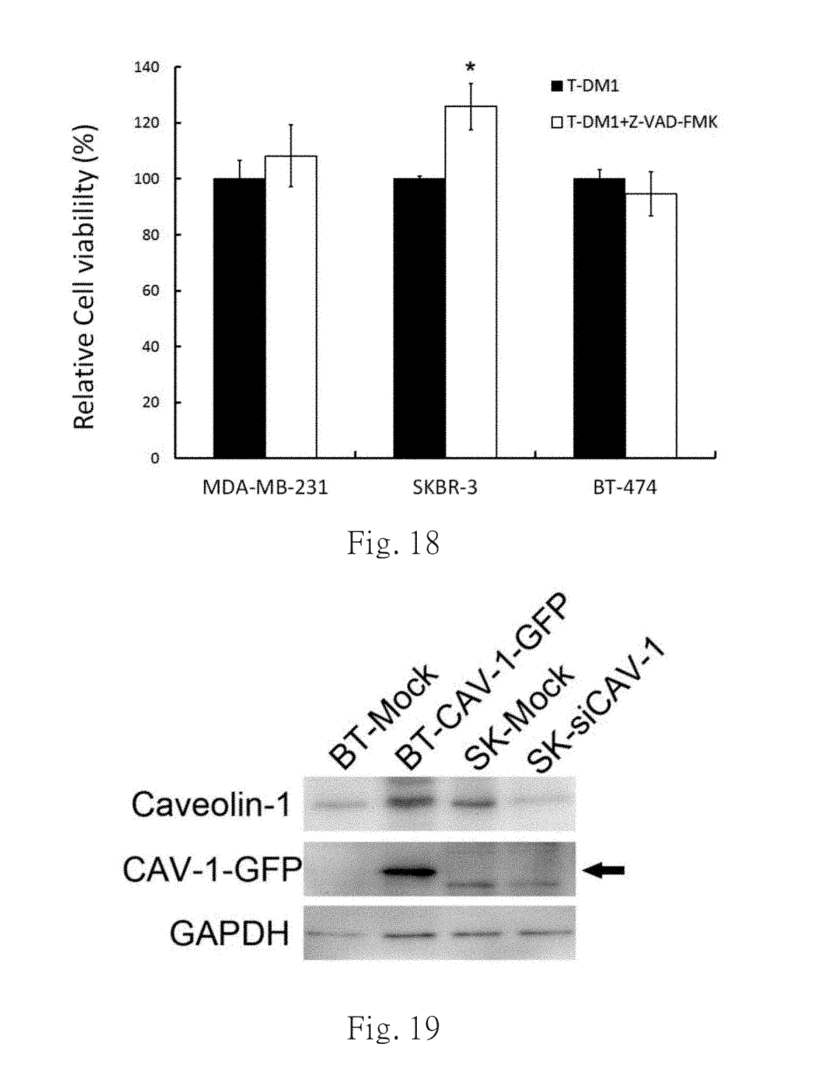

Results from FIGS. 13-17 indicate that lower level of Bcl-2 and increased percentage of apoptosis was detected in T-DM1 treated SKBR-3 cells, whereas fewer necrotic cells were found with PI staining. Furthermore, referring to FIG. 18, drug sensitivity to 1 μg/ml concentration of T-DM1 treatment was decreased when SKBR-3 was treated with pan-caspase inhibitor Z-VAD-FMK, and however, the level of T-DM1 induced cell toxicity may be different dependent on T-DM1 concentration.

These results indicate that HER-2 positive SKBR-3 cells, which have a higher caveolin-1 expression, are sensitive to T-DM1 treatment.

### Example 10: Cell Transfection Assay

BT-474 cells were grown on plates in normal growth medium without antibiotics, and Lipofectamine 2000 transfection reagent (Invitrogen, ThermoFisher, Waltham, Mass., USA) was used to induce GFP-tagged caveolin-1 (Addgene, Cambridge, Mass., USA) overexpression. Caveolin-1siRNA (Dharmacon, GE health care, Lafayette, Colo., USA) was used in the knockdown experiment in SKBR-3 cells.

Cells were analyzed after 24 hour post-transfection, and the efficacy of transfection was confirmed by immunoblot analysis of cell lysates using rabbit anti-GFP (abcam, Cambridge, Mass., USA) and rabbit anti-caveolin-1 (Santa Cruz, Dallas, Tex., USA) antibodies.

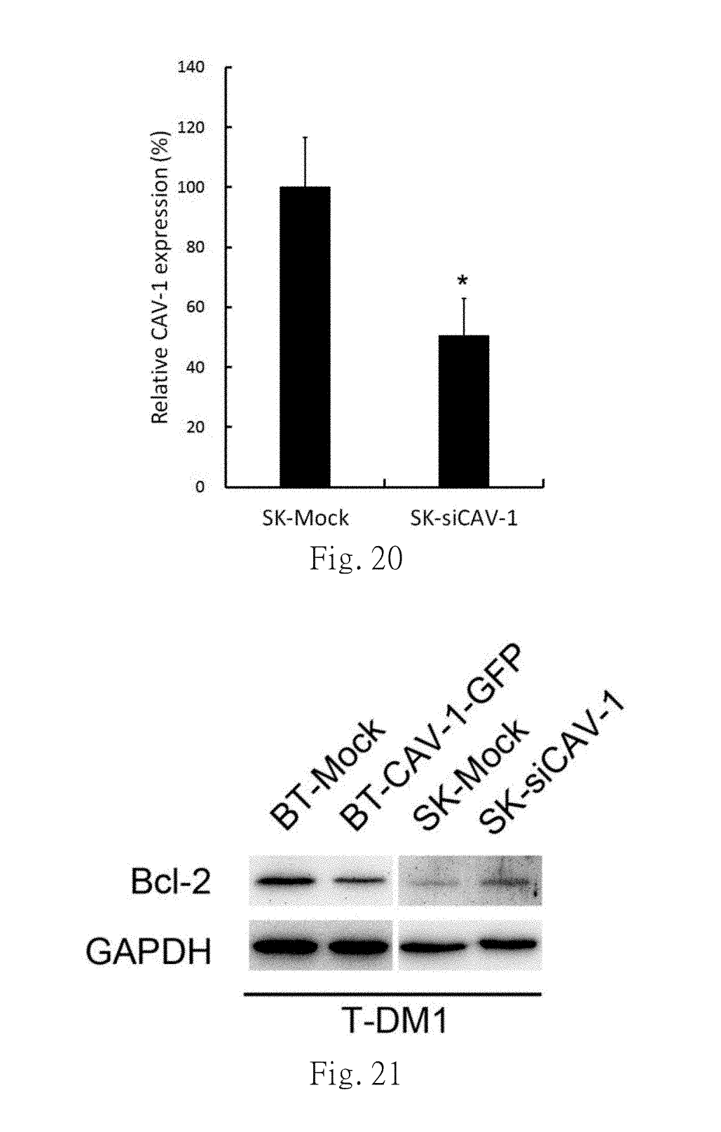

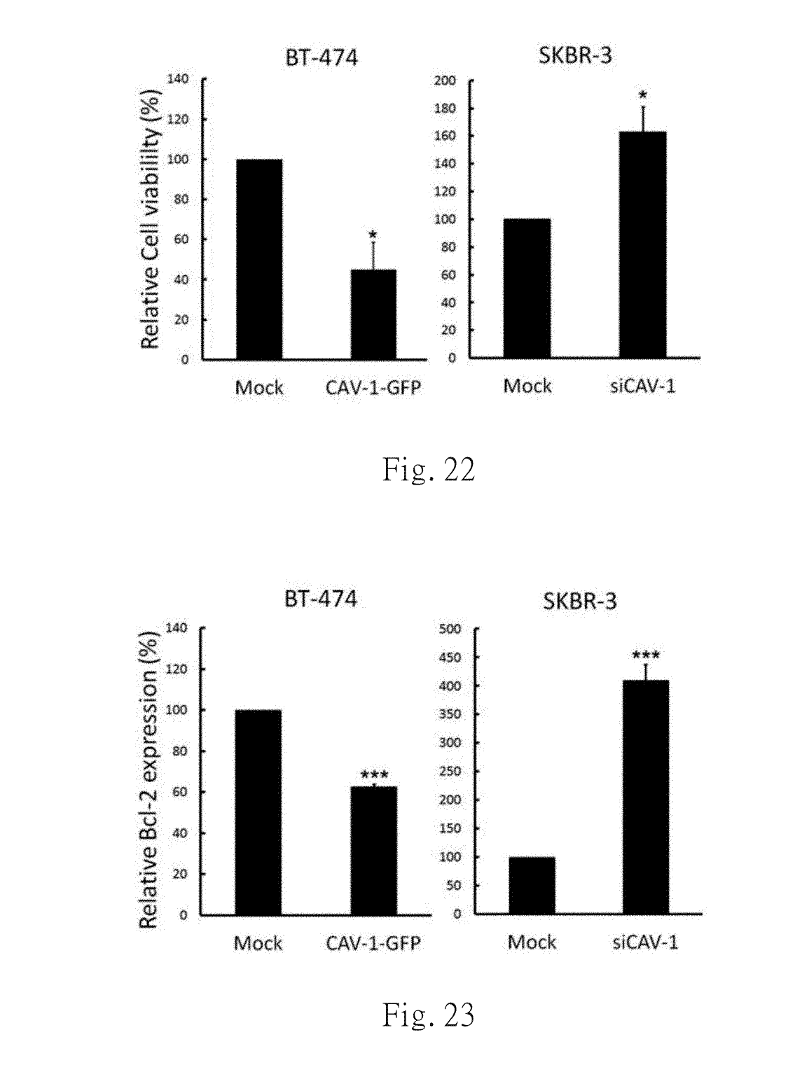

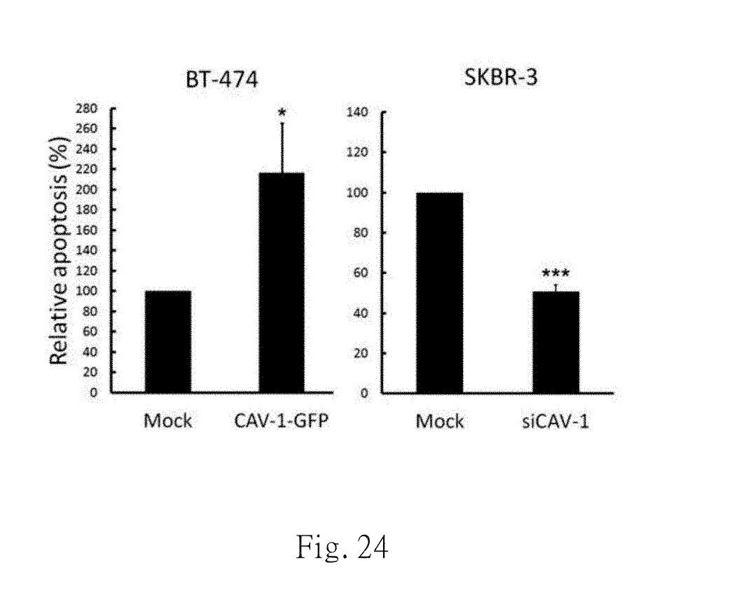

Please referring to FIGS. 19 and 20, results of Western blotting demonstrated that caveolin-1 was overexpressed in GFP-caveolin-1 transfected BT-474 cells, and was suppressed in siRNA-treated SKBR-3 cells. Results from FIG. 21 show that the overexpression of CAV-1 in BT-474 increased T-DM1 cytotoxicity, however, CAV-1 knockdown in SKBR-3 cells significantly reduced T-DM1 efficacy (p<0.05). Furthermore, results from FIGS. 22-24 demonstrate that this T-DM1-induced cytotoxicity was associated with the level of Bcl-2 expression. When the expression levels of CAV-1 increased, Bcl-2 levels were lowered to decrease the cell viability percentage and increase the percentage of apoptosis.

According to these results, the cytotoxic effect of T-DM1 was negatively associated with Bcl-2 levels in both SKBR-3 and BT-474 cells, which express different levels of CAV-1. The percentage of apoptosis measured by flow cytometry was associated with the chemosensitivity of the cells to T-DM1 induced by the overexpression of CAV-1. Taken together, these data suggest that CAV-1 is required for T-DM1 internalization and that this internalized conjugate further induces cell apoptosis.

Through the above-mentioned results of embodiments and examples, the CAV-1 disclosed in this present invention is really associated with sensitivity of cancer target drug. When expression level of CAV-1 is high, endocytosis of cancer target drug will increase to raise cell absorbance percentage of cancer target drug. In other words, the CAV-1 disclosed in this present invention can be a biomarker for assessing drug sensitivity in vitro. More specifically, detecting CAV-1 and Her-2 expression level in a sample provided by a subject can predict the sensitive of target drug in this individual. It not only can raise prognosis but also achieve effect of personal and precise medical treatment. Besides, the biomarker CAV-1 disclosed in this present invention can be the basis for future development of cancer target drug.

What is claimed is:

1. A method for assessing sensitivity of target drug in vitro comprising:
obtaining a tissue sample from a subject; and
determining expression of caveolin-1 in the tissue sample, wherein when the expression of caveolin-1 in the tissue sample is higher than the expression of caveolin-1 in a control sample, the subject is having sensitivity to a cancer target drug.

2. The method according to claim 1, wherein the tissue sample expresses the Her2.

3. The method according to claim 1, wherein the target drug is antibody-drug conjugate (ADC).

4. The method according to claim 2, wherein the target drug is trastuzumab emtansine (T-DM1).

5. The method according to claim 1, including the following steps:
step A: obtaining a normal tissue sample and a Her2-positive breast cancer tissue sample from the subject;
step B: determining expressions of caveolin-1 of the normal tissue sample and the Her2-positive breast cancer tissue sample, respectively;
step C: comparing the expressions of caveolin-1 in the Her2-positive breast cancer tissue sample with the expressions of caveolin-1 in the normal tissue sample, wherein greater levels of caveolin-1 in the Her-2 positive breast cancer tissue as compared to the normal tissue sample is indicative of sensitivity to the target drug in the subject

6. The method according to claim 5, wherein step B is using immunohistochemistry method to determine the expression caveolin-1 in each of the samples.

7. The method according to claim 5, wherein step B is using protein quantification method to determine the expression of caveolin-1 in each of the samples.

8. A biomarker for assessing drug sensitivity in vitro is caveolin-1.

9. A biomarker according to claim 8, wherein the drug is an antibody-drug conjugate (ADC) for breast cancer.

10. A biomarker according to claim 9, wherein the drug is trastuzumab emtansine (T-DM1).
